(19) United States
(12) Patent Application Publication
(10) Pub. No.: US 2007/0270282 A1
(43) Pub. Date: Nov. 22, 2007

(54) Product for Measuring the Effectiveness and Efficiency of Warming-Up and Winding-Down Physical Exercises and Training Equipment Comprising Said Product

(76) Inventor: Deolo Falcone, Francavilla al Mare (IT)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 10/586,519

(22) PCT Filed: Jan. 18, 2005
(86) PCT No.: PCT/IT05/00021

§ 371 Date: Apr. 9, 2007

(30) Foreign Application Priority Data
Jan. 19, 2004 (IT) CH2004A00005
Jan. 19, 2004 (IT) CH2004A00006

(51) Int. Cl. A61B 5/01; G08B 23/00

(52) U.S. Cl. 482/8; 600/549; 340/573.1

(57) ABSTRACT

Product and training equipment comprising said product, to measure the effectiveness and efficiency of warming-up or winding-down physical exercises performed by an individual characterized in that it comprises a temperature sensor to read the body temperature of said individual and means to monitor a variation in the body temperature of said individual produced as a result of said exercises compared to said temperature readings.

The present invention relates to the technical field of training equipment and more particularly to a product for measuring the effectiveness and efficiency of warming-up and winding-down physical exercises and training equipment comprising said product.

The training equipment currently available on the market, regarding preliminary warming-up and/or subsequent winding-down activities, performed during activities of indoor physical training, fitness, wellness and home fitness training, are commercialized:

without instruments to measure the effectiveness of warming-up and/or winding-down activities, leaving said processes to the technical/specialist preparation of the experts in charge of the equipment and to the usual training/winding-down protocols which in some cases form an integral part of the instructions for use provided with the equipment; or

providing the use of cardio frequency meters so that, through detection of the heart rate frequency of the person subjected to physical exercise and its processing by means of special formulae, it is possible to assess the intensity of the physical effort and, above all, the effects which this has on the body.

This latter type of equipment assesses the intensity of the effort generally on the basis of measurement of:

the rate at which the subject's heart beats normally during daily activities;

the contraction heart rate during exposure to physical effort;

and it is further based on the interpretation of said values, on the basis of specific formulae.

Some of the most significant examples of measurements and formulae adopted by people who perform warming-up/winding-down and/or training exercises using a cardio frequency meter are given hereunder.

A typical measurement is given by the Max HR, meaning the threshold level above which effort associated to physical exercise could be harmful; it is generally calculated by means of the formula Max HR=220−age.

Limited to exercise of the “Interval Training” type, in which the person nearly always trains near the aerobic threshold and takes the heart rate close to the upper limit of the aerobic range for a limited time, the parameters detected after training and the special formulae needed to process and therefore to read the instrumental data are, for example, parameters calculated as percentage values of the peak heart rate.

Even if warm-up/wind-down activities are commonly considered essential to carry out any form of physical activity or sport in order to minimize injuries and improve performance, the routines with which athletes attempt to warm-up/wind-down are not always correct: many injuries, among which not only those involving muscles (sprains, contractures, etc.), are caused by incorrect warm-up/wind-down activities (Lehmann et al 1970, Sapega et al 1981).

Correct warm-up/wind-down exercises, thanks to the variation in muscular viscosity, to the capacity to vary the formation of lactate at the beginning/end of performance and to the action of facilitating the neuromotor pattern, (with possible reduction in the disproportionate use of the antagonistic muscles), are fundamental to reduce such injuries.

Warming-up and winding-down activities are definitely indispensable for those who do indoor training, fitness, wellness and/or exercises using training equipment for domestic use (home fitness), during which the intensity of the lesson is not so high as to require indispensable pre/post competition preparation.

It is very important for the people participating in these lessons, both students and instructors, to go through an initial/final phase of the lesson aimed at warming-up/winding-down.

However, even if the instructors are well aware of this (normally, for example, the choreography of aerobics foresees an initial warm-up/final wind-down phase), the students do not always understand and tend to overdo things at the beginning/end: even more dangerous is the situation where the student carries out physical exercise independently (running on the treadmill or using other ergometers) and does not take into due consideration the importance of a gradual, progressive increase/decrease in the intensity of effort, in order to warm-up/wind-down his organism.

From the above, it is obvious that the most critical reason for insufficient improvement in an athlete's performance and for injuries during training, due to incorrect warm-up/wind-down activities, cannot be attributed to the lack of warm-up/wind-down routines but to the lack of knowledge concerning them by those people who do indoor training, fitness, wellness and/or exercises using training equipment for domestic use (home fitness) and who, therefore, do not carry out the requirements of said routines.

For example, in the case where the warm-up routine is based on an initial short run, this is usually carried out at a rhythm which has little influence on the body temperature and normally involves only running forward in a straight line: a deeper analysis shows how this type of preparation for an athlete performing sporting activities is highly limited.

In the same way, the lack of specific knowledge, concerning both the formulae used to process the values recorded by the cardio frequency meter and the complex interaction between the measured heart rate and multiple external factors, limits the possibility of athletes without specific medical knowledge to use said instruments correctly. For example, it is sufficient to consider the following:

in spite of the apparent simplicity of the automatic processing of the data recorded by the cardio frequency meters by mean of multiple algorithms, the values obtained are valid only in standard situations while they cannot be personalized without taking into consideration specific knowledge, since not only is heart rate characteristic for each individual but it also varies according to his age and, above all, to how fit that individual is (for example: Max HR=220−age);

the effort threshold is different for each person: for one individual, simply walking could be sufficient effort to lead to a heart rate of 135 beats per minute, while for another the same effect could be generated by the effort of running for 4 minutes;

emotional conditions (states of anxiety and nervousness, for example linked to the menstrual cycle) also cause alterations in heart rate.

Another element not to be overlooked is the difficulty of any athlete to be aware of his instant heart rate which is characteristic of a training session, while performing said physical activities.

The object of the present invention is to provide a product and equipment which can validate warming-up and winding-down activities by means of a method which is simple to put into effect, is very reliable from the technological point of view, scarcely influenced by external parameters and whose results can be easily interpreted by athletes with no specific medical knowledge, with the aim to limit injuries resulting from incorrect performance of said activities and to improve athletes' performance. Said object is reached by means of a product and by means of equipment as defined in the attached independent claims.

In short, the present invention, of which an exemplary but non-limiting embodiment is described hereunder, provides:

measurement of the athlete's body temperature before and after warm-up/wind-down activities in order to quantify the variation in body temperature generated as a result of said activities;

electronic processing of the measurement readings (based on specific studies carried out by the inventor in said areas with the aim to evaluate the variations in temperature found on examining various men and women, depending on the quantity of warm-up/wind-down activities, for the multiple combinations of: type of physical activity, training equipment, person), in order to inform the athlete when he has reached the required conditions on concluding said activities correctly.

A preferred but non-limiting embodiment of the present invention consists in a control console intended to be mounted on gym equipment, fitted with:

a temperature sensor suitably protected so as to prevent contamination by external agents;

a system to start up/end a temperature measurement cycle;

an output graphic interface to display temperature readings;

a push button panel to enter input data;

a programmable electronic board to manage input and acquisition cycles.

With regard to the operational method, in detail, the preferable but not sole functioning cycle of the invention is composed of the following steps:

insertion of batteries;

switching on the system;

starting up the body temperature detection system and reading of the measurement at the start of activity by means of placing a part of the athlete's body near the sensor;

carrying out the warming-up/winding-down physical activities;

starting up the body temperature detection system and new measurement of the temperature by means of placing a part of the athlete's body near the sensor.

Further features and advantages of the present invention will become more apparent from the following detailed description of an exemplary but non-limiting embodiment thereof, as illustrated in the accompanying drawings, in which:

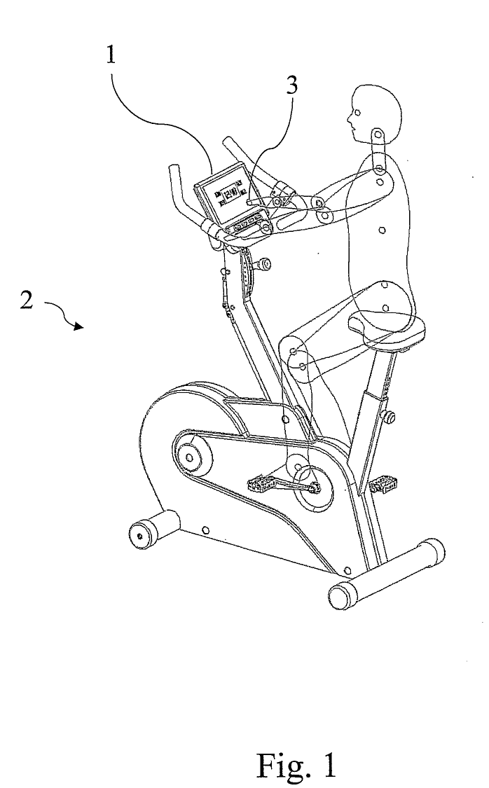

FIG. 1 shows a view of the functioning of a product according to the invention and of training equipment including said product;

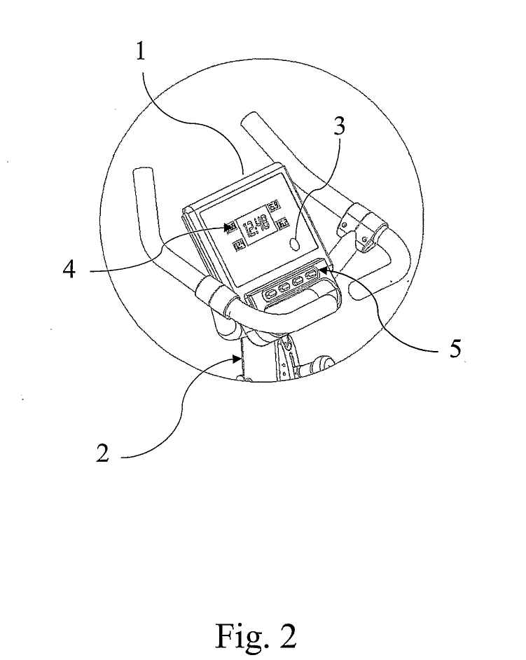

FIG. 2 shows the product and equipment of FIG. 1 in greater detail.

In the figures, equal or similar elements are indicated with the same reference numbers.

With reference to the attached figures, 1 indicates a device or product for measuring the effectiveness and efficiency of preliminary warming-up or subsequent winding-down activities, performed during activities of indoor physical training, fitness, wellness and/or home fitness training. Advantageously, said product 1 is based on monitoring the body temperature of the person performing said activity.

In the preferred embodiment in FIG. 1 said product forms part of the training equipment 2, which in this embodiment is an exercise cycle 2.

The product 1 is able to quantify a variation in temperature produced after said activity by means of measuring the athlete's body temperature before and during the exercises, based on the multiple combinations of types of physical activity/training equipment.

The product or device 1 is also able to process the temperature readings, depending on the specific controls, in order to inform the athlete when he has reached the conditions required by the training session.

In the preferred embodiment shown in FIGS. 1 and 2, the product 1 consists in a control console 1, mounted on training equipment, fitted with:

a temperature sensor 3 (in the embodiment a thermocouple) suitably protected so as to prevent contamination by external agents;

connections;

an output interface 4 which in the embodiment is a graphic screen which permits display of temperature readings;

a push button panel 5 intended for insertion of input, including a command to select start-up of temperature measurement and a command to switch on/off the apparatus;

a programmable electronic card for management of input and acquisition cycles;

a compartment for the power supply system of the invention.

In a particularly preferred embodiment, said control console is equipped with an independent power supply by means of batteries placed in the lower part of the console, contained in a special compartment closed with a screw-on top.

With regard to operation, the product or device 1 preferably functions according to the following steps:

system start-up;

activation of the body temperature detection system and reading of the measurement at the beginning of the session, by means of placing one of the athlete's fingers near the sensor (the choice of a finger as the part of the body for the reading is not binding);

performance of physical activity;

activation of the body temperature detection system and reading of the new measurement after the session, by means of placing a finger near the sensor once again.

The product or device 1 according to the invention can be produced in various forms, by using different temperature sensors (for example invasive or non-invasive, contact or no-contact, etc.) and/or a combination of them or in different positions.

Moreover, in an embodiment variation, said product can be produced with temperature measurement systems which measure the athlete's temperature “continuously” and/or “at intervals”, as well as by previous specific input of this data, as provided in the embodiment described above.

In a different embodiment, the product 1 can also be produced without the output interface 4 to display the temperature readings, but which indicates when the athlete has reached the set minimum temperature variation by various kinds of signals (sound, optical, etc.).

Preferably, the product 1 can be produced with its own power supply (by means of using a dynamo, photovoltaic cells, etc.). Alternatively, an external power supply can be provided.

Moreover, in different embodiments, the product or device 1 is able to measure the temperature of body parts other than the finger since the choice of this as the body part on which to carry out measurement is not binding: in fact, following numerous analyses and tests, it was noticed that all parts of the body had a similar increase in temperature due to the effects of the body's own heat regulation, even if at the beginning of warm-up, the temperature of the extreme parts decreases as a result of the blood flowing to the muscles being used in the exercises.

In a particularly preferred embodiment, the temperature sensor used is a sensor of the “no-contact” type, for example an infrared temperature detector.

It should be observed that the temperature sensor (3) need not necessarily be mounted on the control console of the training equipment. For example, if said equipment includes a handle (or handgrip), the sensor can be mounted on said handle, for example near a special projection provided on said handle.

In a particularly advantageous embodiment, the product or device according to the present invention is able to inform the user when he has reached a correct stage of warm-up/wind-down, when by measuring the variation in body temperature and comparing it to the temperature at the beginning of the exercises, said variation reaches an absolute value in the range of approximately 1.3° C.-2.3° C. and preferably in the range of 1.5° C.-2.0° C., more preferably equal to approximately 1.7° C.

In a further advantageous embodiment, if the product is included in training equipment it is able to modify, on the basis of variations in temperature detected after said training, some parameters of the training routine with the aim of reaching a pre-established temperature variation and depending on the type of physical activity and the individual concerned. For example, said parameters include training times and the power developed by the individual in the unit time or speed.

It should be noted that a product or device 1 according to the invention can also be produced as a system not intended for training equipment or apparatus but as a unit in itself (for example, in the form of a bracelet, personal accessory, etc.).

The advantages connected to the use of said product or device 1 are numerous and consist in:

its intuitive use since validation of warm-up activities only depends on temperature variation: the data detected by the instrument do not have to be processed by means of special formulae and do not require special knowledge to be correctly interpreted;

limited influence of external factors on the system monitoring the body temperature of the person performing physical activities;

greater reliability and solidity of the product compared to cardio frequency meters, given the cardio-mechanical nature of the product.

the possibility of providing supplementary equipment for all existing gym apparatus, even those already on the market, without the need for particular refitting operations;

the possibility of being produced in multiple variations which can easily be improved in accordance with the technological evolution of the data collection/processing systems and electronic analysis logics.

reduced production costs;

more time available for training thanks to reduced times necessary for warming-up resulting from the possibility of validating that it is performed correctly: in fact, presuming that, due to inadequate warm-up, the athlete needs a further 10-15 minutes to warm-up correctly during the main part of the training session, the same amount of time could be saved during each training session. If the athlete trains 3 times per week for 40 weeks (one year of training), there could be a total increase in training time of 20-30 hours per year, to the detriment of supplementary warming-up/winding-down activities;

the possibility of being put on the market as an “independent” system to measure the effectiveness and efficiency of warm-up/wind-down activities, irrespective of the gym apparatus.

Moreover, a product or device or training equipment according to this invention can advantageously be used during training by individuals who perform said activities without the necessary technical competence and/or without specific supervision by suitably trained staff.

Naturally, in order to satisfy contingent and specific requirements, a person skilled in the art may apply to the above-described product or device and training equipment according to the invention many modifications and variations, all of which, however, are included within the scope of protection of the invention as defined by the following claims.

1. Product to measure the effectiveness and efficiency of warming-up and winding-down physical exercises performed by an individual characterized in that it comprises a temperature sensor to detect the body temperature of said individual and means of monitoring, by comparing said temperature readings, variations in the body temperature of said individual as a result of said exercises.

2. Product according to claim 1, wherein said monitoring means include electronic processing means to process said temperature readings.

3. Product according to claim 1, wherein said temperature sensor includes a thermocouple.

4. Product according to claim 1, wherein said temperature sensor is of the no-contact type.

5. Product according to claim 4, wherein said temperature sensor is an infrared sensor.

6. Product according to claim 1, further comprising protection means to prevent contamination of said temperature sensor by external agents.

7. Product according to claim 1, further comprising an output interface to display said temperature readings.

8. Product according to any of the previous claims, further comprising means to indicate when the individual has reached the pre-established training conditions.

9. Product according to claim 1, wherein said temperature is measured continuously.

10. Product according to claim 1, wherein said temperature is measured at intervals.

11. Product according to claim 1, further comprising control means to control the beginning and the end of a cycle of said measurements.

12. Product according to claim 1, wherein said sensor is able to read said temperature measurements by placing a body part of said individual near or on said sensor.

13. Product according to claim 12, wherein said sensor is able to read said temperature measurements by placing a finger of said individual near or on said senor.

14. Product according to claim 1, wherein said measurement readings include an initial temperature measured at the beginning of said physical activities or at the start of a measurement cycle and where said monitoring is such as to monitor a difference in temperature of said readings compared to the initial reading.

15. Product according to claim 1, wherein said product is able to signal achievement of a correct warm-up/wind-down state of said individual when, by means of said monitoring of variations in body temperature compared to the beginning of said activities or at the start of a measurement cycle, said variation reaches an absolute value approximately within the range of 1.3° C.-2.3° C.

16. Product according to claim 15, wherein said range is approximately within 1.5° C. and 2.0° C.

17. Product according to claim 1, wherein said product is able to signal achievement of a correct warm-up/wind-down state of said individual when, by means of said monitoring of variations in body temperature compared to the beginning of said activities or at the start of a measurement cycle, said variation reaches an absolute value approximately equal to 1.7° C.

18. Product according to claim 1, wherein said product is in the form of a control console for training equipment.

19. Product according to claim 1, wherein said product is in the form of a bracelet or personal accessory.

20. Product according to claim 1, further comprising independent power supply means.

21. Training equipment characterized in that it comprises a product according to claim 1.

22. Training equipment according to claim 21, wherein said equipment comprises at least one handle, or handgrip, provided with a projection and wherein said sensor is placed near said projection.
